(19) United States
(12) Patent Application Publication
(10) Pub. No.: US 2025/0359856 A1
(43) Pub. Date: Nov. 27, 2025

(54) COAXIAL NEEDLES AND BIOPSY DEVICES FOR PERFORMING A BIOPSY

(71) Applicant: BARD PERIPHERAL VASCULAR, INC., Franklin Lakes, NJ (US)

(72) Inventors: Jordan ADDISON, Gilbert, AZ (US); Nicholas MOWREY, Scottsdale, AZ (US); Heather A. STORM, Phoenix, AZ (US)

(73) Assignee: BARD PERIPHERAL VASCULAR, INC., Franklin Lakes, NJ (US)

(21) Appl. No.: 19/109,004

(22) PCT Filed: Sep. 9, 2022
(86) PCT No.: PCT/US2022/076166

§ 371 Date: Mar. 5, 2025

(51) Int. Cl. A61B 10/02

(52) U.S. Cl. CPC A61B 10/0233

(57) ABSTRACT

A coaxial needle for performing a biopsy procedure includes a cannula and a stylet. The cannula has a proximal end and a distal end, and a first inflatable member surrounds the cannula in a circumferential direction from the proximal end to the distal end. The first inflatable member applies an outward pressure along the entire length of the first inflatable member in an inflated state.

### TECHNICAL FIELD

The present disclosure relates to coaxial needles, biopsy devices, and methods, and, more particularly, to coaxial needles, biopsy devices, and methods of performing a biopsy procedure that includes securing the coaxial needle in place.

### BACKGROUND

A typical percutaneous needle biopsy may involve the use of a coaxial introducer needle to gain access to a biopsy site through the surrounding soft tissue, such as skin, subcutaneous tissue, and organ tissue. The use of the coaxial needle may allow a user to utilize the same needle tract to the biopsy site for multiple tissue sample acquisitions, thereby minimizing the potential damage to the surrounding tissue. However, during biopsy procedures, the coaxial needle may begin to back out of the needle tract, which may result in bleeding and other complications. In some devices used for biopsy procedures, small amounts of compressed hydrogen or argon gas may be released at the needle tip to freeze the needle within the biopsy tract in order to prevent the coaxial from backing out or moving. However, the compressed hydrogen or argon often results in additional damage to the tissue in the biopsy tract. Furthermore, the process of freezing the needle employed by conventional biopsy devices is difficult to reverse, which can make adjustment of the coaxial difficult.

### SUMMARY

An object of the present disclosure is to provide a coaxial needle that can be inflated in a preferential manner in order to secure the coaxial needle in a biopsy tract during biopsy procedures.

In one embodiment, a coaxial needle may include a cannula having a distal end and a proximal end, and a first inflatable member surrounding the cannula in a circumferential direction and having a length extending from the distal end to the proximal end of the cannula. The first inflatable member may apply an outward pressure along its entire length in an inflated state.

In another embodiment, a coaxial needle may include a cannula having a distal end and a proximal end, and a first inflatable member surrounding the cannula in a circumferential direction and having a length extending from the distal end to the proximal end of the cannula. The first inflatable member may further have a first inflated state and a first deflated state. The coaxial needle may further include a stylet including an elongate member having a first end and a second end. The stylet may also have a second inflatable member coupled to the second end of the elongate member, and the second inflatable member may have a second inflated state and a second deflated state.

In yet another embodiment, a method of performing a biopsy is disclosed. The method may involve inserting a coaxial needle into a target site, the coaxial needle including a cannula having a proximal end and a distal end. The method may further involve inflating a first inflatable member which surrounds the coaxial needle in a circumferential direction and extends from the proximal end of the cannula to the distal end of the cannula. A biopsy needle may be inserted in the cannula of the coaxial needle to acquire sample from the target site, such that a biopsy void is created in the target site. Once the samples have been obtained, the method may involve removing the biopsy needle from the cannula of the coaxial needle. A stylet including an elongate member having a first end and a second end may then be inserted in the cannula of the coaxial needle. The method may further involve inflating a second inflatable member located on the second end of the elongate member of the stylet in the biopsy void created in the target site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a perspective view of a coaxial needle assembly, according to one or more embodiments shown and described herein;

FIG. 2 depicts a longitudinal side view of the coaxial needle assembly of FIG. 1, according to one or more embodiments shown and described herein;

FIG. 3A depicts a longitudinal side view of a cannula of the coaxial needle of FIG. 1 in a deflated state, according to one or more embodiments shown and described herein;

FIG. 3B depicts a longitudinal side view of the cannula of FIG. 3A in an inflated state, according to one or more embodiments shown and described herein;

FIG. 4 is a block representation of the pressure source assembly of FIGS. 3A-3B;

FIG. 5 depicts a longitudinal side view of an embodiment of a cannula of FIG. 1 in an inflated state, according to one or more embodiments shown and described herein;

FIG. 6 depicts a longitudinal side view of an embodiment of a cannula of FIG. 1 in an inflated state with a biopsy needle, according to one or more embodiments shown and described herein;

FIG. 7A depicts a longitudinal side view of a second inflatable member of a stylet of the coaxial needle of FIG. 1 in a deflated state, according to one or more embodiments shown and described herein;

FIG. 7B depicts a longitudinal side view of the second inflatable member of a stylet of the coaxial needle of FIG. 1 in an inflated state, according to one or more embodiments described herein; and

FIG. 8 depicts a flow diagram of an illustrative method for performing a biopsy procedure using the coaxial needle of FIG. 1, according to one or more embodiments shown and described herein.

Reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to coaxial needles, biopsy devices and methods of performing biopsy procedures. For example, in embodiments, a coaxial needle includes a cannula defining a distal end and a proximal end, and a first inflatable member surrounding the cannula in a circumferential direction and having a length extending from the distal end to the proximal end of the cannula. In some embodiments, the coaxial needle may be transitioned between a deflated state and an inflated state such that the first inflatable member applies an outward pressure along its entire length in the inflated state. In the inflated state, the outward pressure applied by the first inflatable member along its length may act to anchor the coaxial needle within a biopsy tract.

Embodiments of the present disclosure may be specifically advantageous for securing the coaxial needle in a desired anatomical position without risk of the needle backing out or moving along the biopsy. Once the coaxial needle is positioned in the desired anatomical position, the coaxial needle may be transitioned from the deflated state to the inflated state, such that the first inflatable member applies outward force along the biopsy tract at the desired anatomical position. This outward pressure may act to secure the coaxial needle within the biopsy tract. Furthermore, the pressure applied by the inflatable member of the coaxial needle on the biopsy tract may aid in hemostasis. The hemostatic properties of the coaxial may be further bolstered by coating the inflatable member with fibrin, starch, collagen, gelatin, polyvinyl acetate (PVA), or other hemostatic agents.

In some embodiments, the coaxial needle may further include a stylet having a first end and a second end. In such embodiments, a second inflatable member may be located on the distal end of the stylet. The stylet may be sized to fit inside the cannula. The stylet may be inserted through the cannula of the coaxial after a tissue sample has been removed from the biopsy site. In these embodiments, the second inflatable member may be transitioned between a second deflated state and a second inflated state, such that the second inflatable member applies an outward pressure in the second inflated state. The second inflatable member may further conform to a void left by the removal of the tissue sample at the biopsy site in the second inflated state. The outward pressure applied by the second inflatable member to the void left by the removal of the tissue sample at the biopsy site may act to prevent bleeding at the biopsy site.

Embodiments of coaxial needles, biopsy devices, and methods of performing biopsy procedures will now be described in more detail herein with reference to the drawings and where like numbers refer to like structures.

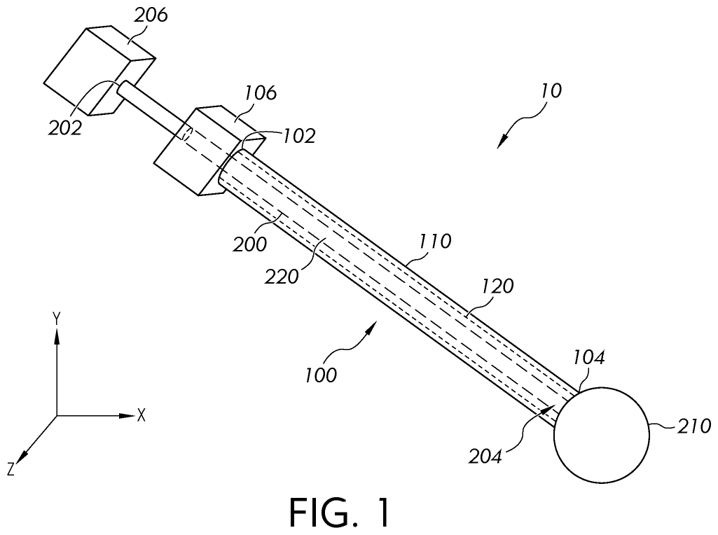

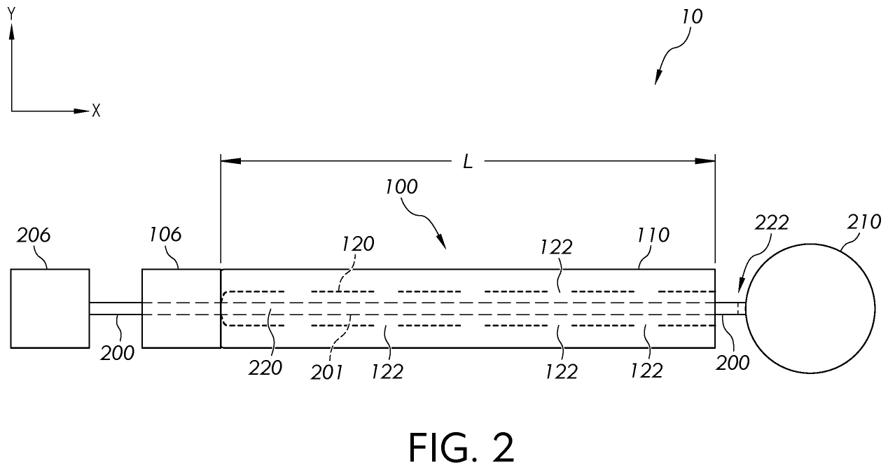

Referring now to FIGS. 1 and 2, a coaxial needle assembly 10 is depicted. The coaxial needle assembly may include a cannula 100 having a first inflatable member 110 and a stylet 200 having a second inflatable member 210. A greater or fewer number of components may be included without departing from the scope of the present disclosure

The cannula 100 defines a proximal end 102 and a distal end 104. In some embodiments, the distal end 104 may be a sharpened distal end, such that the cannula 100 may form a biopsy tract as the cannula is inserted into a patient. The cannula 100 may define an inflation lumen 120 extending through the length of the cannula 100, which may be fluidly coupled to the first inflatable member 110, described in greater detail below. Extending through the cannula 100 from the first inflation lumen 120 may be one or more inflation openings 122, such as a plurality of inflation openings 122 which may act to fluidly couple the inflation lumen 120 to the first inflatable member 110. The first inflatable member 110 may surround the cannula 100 in a circumferential direction and have a length L which may extend from the distal end 104 to the proximal end 102 of the cannula 100. The first inflatable member 110 may extend over each of the one or more inflation openings 122 in the inflation lumen 120, such that the introduction of fluid into inflation lumen 120 may result in the first inflatable member 110 being inflated. In some embodiments, the first inflatable member 110 may be inflated with a fluid such as air, saline, or any other suitable gas. In some embodiments, the inflation lumen 120 may be coupled to the cannula 100 via any suitable connection (e.g., via threaded connection, adhesive, welding, brazing, etc.). As illustrated in FIGS. 1 and 2, the cannula 100 may have a round cross-sectional shape, although it should be understood that the cannula 100 may have any other suitable cross-sectional shape (e.g., rectangular).

The coaxial needle assembly 10 may further include a stylet 200 which may be insertable through the cannula 100. In some embodiments, the stylet may have a length Ls which is greater than the length L of the cannula, such that the stylet 200 may extend through the cannula 100 and beyond the distal end 104 of the cannula 100. The stylet 200 may generally include an elongate member 201 having a first end 202 and a second end 204. In such embodiments, the cannula 100 may have an inner diameter which is larger than an outer diameter of the stylet 200, such that the stylet 200 may be inserted through the cannula 100 and into a target site. The stylet 200 may include a stylet inflation lumen 220 which extends through the length of the elongate member 201 from the first end 202 to the second end 204 and defines a stylet inflation lumen 220. The stylet inflation lumen 220 may further include an inflation hole 222 positioned on the second end 204 in fluid communication with the stylet inflation lumen 220.

Coupled to the second end 204 of the elongate member 201 may be a second inflatable member 210. The second inflatable member may be fluidically coupled to the stylet inflation lumen 220 of the elongate member 201 via the inflation hole 222. For example, the second inflatable member 210 may be affixed to the second end 204 of the elongate member 201 such that the second inflatable member 210 covers the inflation hole, which may allow the second inflatable member 210 to be selectively inflated when fluid is introduced into the stylet inflation lumen 220. The second inflatable member may be inflated with a fluid such as air, saline, or any other suitable gas. Further, the second inflatable member may be coupled to the second end 204 of the second inflatable member 210 via any suitable connection (e.g., via threaded connection, adhesive, welding, brazing, etc.). As illustrated in FIGS. 1 and 2, the second inflatable member 210 may have a round cross-sectional shape, although it should be understood that the second inflatable member 210 may have any other suitable cross-sectional shape. In these embodiments, the cannula 100 may have an inner diameter which is larger than an outer diameter of the stylet 200, such that the stylet 200 may be inserted through the cannula 100 and into a target site 350.

Referring still to FIGS. 1 and 2, the first inflatable member 110 and/or second inflatable member 210 may be formed from typical materials including polymers such as polyethylene terephthalate (PET), polyetherimide (PEI), polyethylene (PE), polytetrafluoroethylene (PTFE), expanded polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), poly ether-ester, polyester, polyamide, elastomeric polyamides, block polyamide/ethers, polyether block amide, silicones, Marlex high-density polyethylene, Marlex low-density polyethylene, linear low density polyethylene, polyetheretherketone (PEEK), polyimide (PI), polyphenylene sulfide (PPS), polyphenylene oxide (PPO), polysulfone, nylon, perfluoro (propyl vinyl ether) (PFA), other suitable materials, or mixtures, combinations, copolymers thereof, polymer/metal composites, and the like. The first inflatable member 110 and/or second inflatable member 210 may be non-compliant, such that the members maintain their size and shape in one or more directions when inflated. However, the first inflatable member 110 and/or second inflatable member may be semi-compliant or compliant instead. It should be understood that the material used for forming the first inflatable member 110 and/or second inflatable member 210 may impact the compliancy of the inflatable members. Furthermore, it should be understood that while the coaxial needle assembly 10 may include both the first inflatable member 110 and the second inflatable member 210, the coaxial needle assembly 10 may similarly include one of the first inflatable member 110 and the second inflatable member 210 without departing from the scope of the present application.

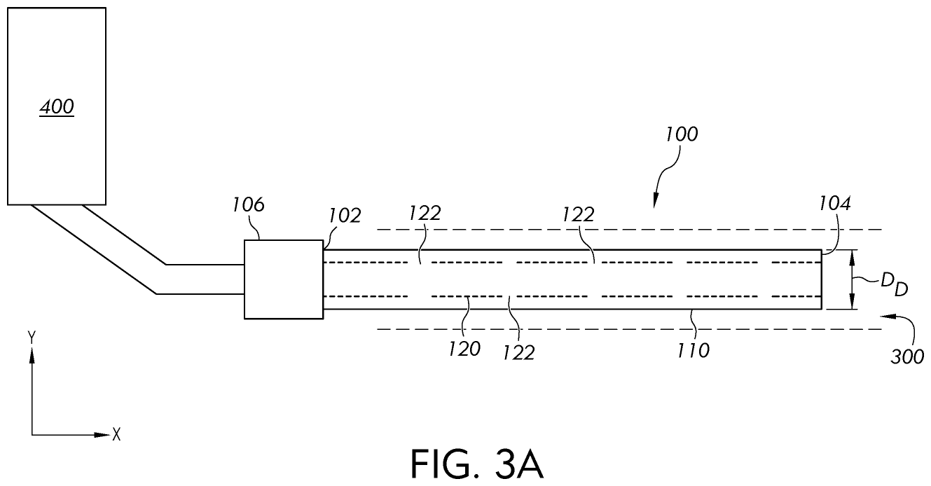

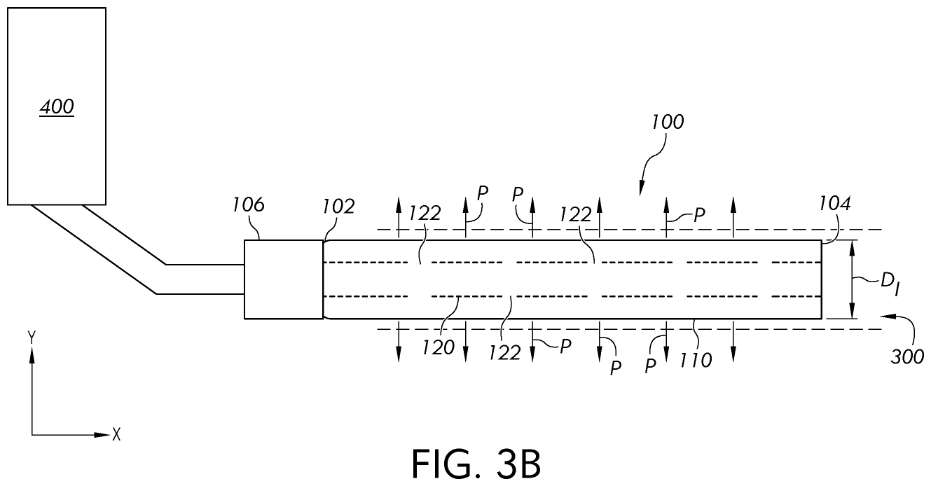

Turning now to FIGS. 3A-3B, the cannula 100 of the coaxial needle assembly 10 is illustrated in a deflated state and an inflated state, respectively, in isolation from the stylet 200. As shown in FIG. 3A, the deflated state may be defined as a pre-deployed state (e.g., the state of the cannula 100 prior to inflation of the first inflatable member 110). In the deflated state, the first inflatable member 110 of the cannula 100 may be deflated, such that the cannula may be easily inserted and positioned in a biopsy tract 300. In these embodiments, the cannula 100 may have a first outer diameter D_{D} in the deflated state (FIG. 3A), and a second outer diameter D_{I} in the inflated state (FIG. 3B), such that the second outer diameter is greater than the first outer diameter.

With the cannula 100 positioned in its desired location within the biopsy tract, the first inflatable member 110 may be inflated, as illustrated in FIG. 3B. In these embodiments, the proximal end 102 of the cannula 100 may be connected to an inflation port 106, which may provide fluid to the inflation lumen 120 of the cannula 100. In some embodiments, the proximal end 102 of the cannula 100 may be molded to the first inflation port 106. In other embodiments, the proximal end 102 of the cannula 100 be coupled to the inflation port 106 via any suitable connection (e.g., via threaded connection, adhesive, welding, brazing, etc.).

Referring still to FIG. 3B, the first inflation port 106 may be used to fill the inflation lumen 120 with fluid. As the fluid fills the inflation lumen 120, the fluid may pass through the one or more inflation openings 122 positioned along the length of the inflation lumen 120, which in turn may cause the first inflatable member 110 to inflate. As the first inflatable member 110 expands to the inflated state illustrated in FIG. 3B, the first inflatable member 110 may begin to exert an outward pressure P on the biopsy tract 300. In some embodiments, the outward pressure P applied by the first inflatable member 110 may act to anchor the cannula 100 in the biopsy tract, such that the cannula 100 may not move in a longitudinal or rotational direction. Additionally, the outward pressure P applied by the first inflatable member 110 may aid in preventing bleeding within the biopsy tract. As the first inflatable member 110 exerts outward pressure P on the biopsy tract, the pressure may act to stop the flow of blood within the biopsy tract, which may aid in initiating natural clotting processes within the biopsy tract.

In some embodiments, the first inflatable member 110 may be coated with a hemostatic agent to further assist in preventing bleeding within the biopsy tract. In these embodiments, the first inflatable member may be coated with any coating having suitable hemostatic properties, including fibrin, starch, collagen, gelatin, PVA, or the like. It should be understood that the hemostatic coating utilized on the first inflatable member 110 may vary depending on the desired biological result. For example, some hemostatic agents may directly aid in clotting processes, while other hemostatic agents may lead to faster coagulation by absorbing water and blood plasma within the biopsy tract.

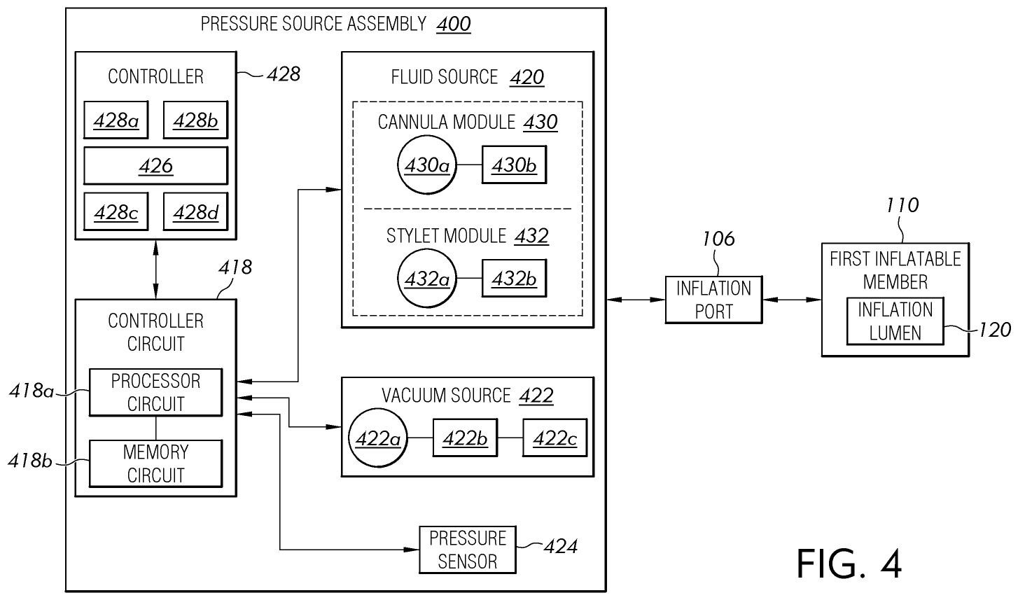

Referring now to FIG. 4, the coaxial needle 10 may further include a pressure source assembly 400, which may alternate the first inflatable member 110 between the inflated state and the deflated state. The pressure source assembly 400 may include a controller circuit 418, a fluid source 420, a vacuum source 422, a pressure sensor 424, and a battery 426. The pressure source assembly 400 may further include a controller 428, such as a computer, which may allow a user to operate the pressure source assembly 400. In these embodiments, the controller 428 may be configured to provide automatic control of the pressure source assembly 400 via a software program, or may be manually controlled via a user manipulating a user interface thereof.

Referring still to FIG. 4, the controller 428 may include control buttons and visual/aural indicators, such as a display and/or speakers, with the control buttons providing user control over various functions of the pressure source assembly 400, and with the visual/aural indicators providing visual/aural feedback of the status of one or more conditions and/or positions of components of the pressure source assembly 400. The control buttons may include one or more buttons for inflating and/or deflating the first inflatable member 428a, 428b and one or more buttons for inflating and/or deflating the second inflatable member 428c, 428d.

Controller circuit 418 is electrically and communicatively coupled to fluid source 420, vacuum source 422, pressure sensor 424, and controller 428, such as by one or more wires or circuit traces. Controller circuit 418 may be assembled on an electrical circuit and includes, for example, a processor circuit 418a and a memory circuit 418b.

Processor circuit 418a has one or more programmable microprocessors and associated circuitry, such as an input/output interface, buffers, memory, etc. Memory circuit 418b is communicatively coupled to processor circuit 418a, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), flash memory, etc. Controller circuit 18 may be formed as one or more Application Specific Integrated Circuits (ASIC).

Controller circuit 418 is configured via software and/or firmware residing in memory circuit 418b to execute program instructions to perform functions associated with the inflation and/or deflation of the first inflatable member 110 of the cannula 100 and/or the second inflatable member 210 of the stylet 200.

Fluid source 420 may include, for example, a cannula module 430 and stylet module 432. Each of the cannula module 430 and stylet module 432 may be electrically and controllably coupled to controller circuit 418. As provided herein, the cannula module 430 and stylet module 432 may be electrically coupled to the controller circuit 418 by way of electrical wiring or any other suitable electrical connections, such that user inputs on the controller 428 may be relayed to the controller circuit 418 and used to control the fluid delivered by the cannula module 430 and/or stylet module 432 respectively. In these embodiments, the cannula module 430 may include a first fluid pump 430a to which a first valve 430b is attached. The stylet module 432 may include a second fluid pump 432a to which a second valve 432b is attached.

Vacuum source 422 may be electrically and controllably coupled to controller circuit 418, and may include, for example, an electric motor 422a that drives a vacuum pump 422b. Vacuum source 22 may further include at least one vacuum port 422c coupled to vacuum pump 422b for establishing vacuum in the first and/or second inflatable member 110, 210.

In these embodiments, the pressure source assembly 400 may be coupled in fluid communication to the inflation port 106, such that the cannula module 430 is able to control the volume of fluid which enters the inflation lumen 120, and in turn, the first inflatable member 110. For example, once the cannula 100 is positioned within the biopsy tract, the first fluid pump 430a may be operated to cause fluid to enter the inflation lumen 120 such that the first inflatable member 110 is inflated to the inflated state. As the fluid is pumped from the first fluid pump 430a into the inflation lumen 120 of the cannula 100, the pressure sensor 424 may monitor the outward pressure P applied by the first inflatable member 110 on the biopsy tract 300. Once the appropriate amount of pressure P has been achieved, the controller circuit 418 may deactivate the fluid pump 430a in order to prevent additional fluid from entering the inflation lumen 120.

In the event that adjustment of the coaxial needle 10 within the biopsy tract is necessary, the vacuum source 422 may be activated to create a vacuum that is usable to remove fluid from the first inflatable member 110. As the fluid is drawn from the first inflatable member 110, the outward pressure P applied on the biopsy tract 300 may decrease, which may in turn act to deflate the first inflatable member 110 such that the cannula 100 is no longer anchored within the biopsy tract 300. When the first inflatable member 110 is deflated, the cannula 100 may be repositioned as necessary. Once the cannula is in the desired position, the first fluid pump 430a may be activated to supply fluid to the first inflatable member 110, such that the cannula becomes anchored in the biopsy tract 300 once more. Similarly, once a biopsy procedure has been completed, the pressure source assembly 400 may alternate the first inflatable member 110 to the deflated state, such that the coaxial needle 10 may be easily removed from the biopsy tract.

In some embodiments, the first inflatable member 110 may be constructed so as to form a predetermined shape in the inflated state. For example, the first inflatable member 110 may be constructed so as to form a cylinder, or any other suitable shape, having a predetermined diameter. In these embodiments, the predetermined shape of the first inflatable member 110 may correspond to the size and shape of the biopsy tract, such that the first inflatable member 110 may anchor the cannula 100 in the biopsy tract in the inflated position. Although the first inflatable member 110 may be made to form a predetermined shape, it should be understood that the shape of the first inflatable member 110 may not be uniform across the entire length of the first inflatable member 110. For example, the predetermined shape of the first inflatable member 110 may include segments of varying diameters, which may be necessary in order to anchor the cannula 100 within the biopsy tract.

In other embodiments, the first inflatable member 110 may be made to conform to the size and shape of the biopsy tract in the inflated state. In these embodiments, the pressure sensor 424 may be used to sense the inflation of the first inflatable member 110. The inflation pressure sensed by the pressure sensor 424 may be relayed to the processor circuit 418a, which may ensure that the first inflatable member 110 may exert an optimized amount of outward pressure P on the biopsy tract. By optimizing the outward pressure P exerted on the biopsy tract, the first inflatable member 110 may maximize the hemostatic benefits provided to the biopsy tract without causing additional expansion of the biopsy tract.

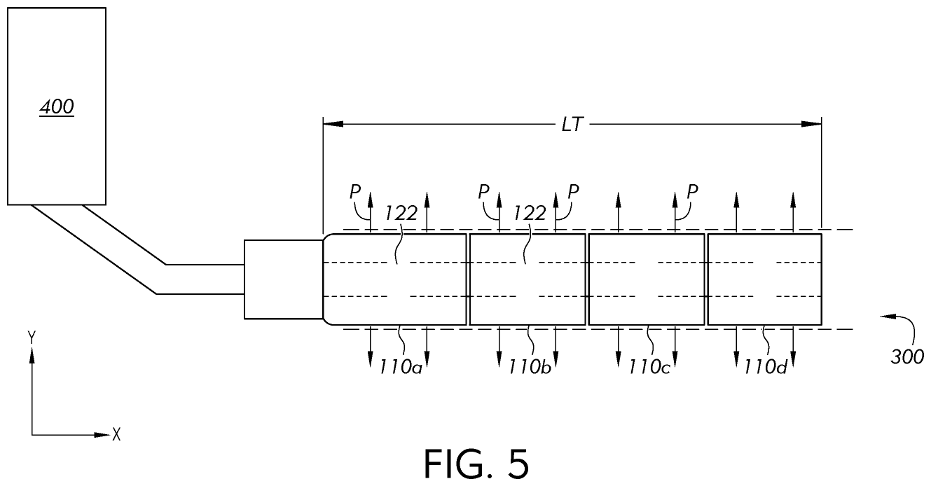

Referring now to FIGS. 4 and 5, a second embodiment of the cannula 100 is illustrated. In this embodiment, the first inflatable member 110 may include a plurality of individual inflatable elements 110a, 110b, 110c, 110d, rather than the single first inflatable member 110 illustrated in FIGS. 3A-3B. As illustrated in FIG. 5, the plurality of individual inflatable elements 110a-110d may extend from the proximal end 102 to the distal end 104 of the cannula 100, such that the total length LT of the individual inflatable elements 110a-110d may equal the length L of the single inflatable member 110 illustrated in FIGS. 3A-3B. In some embodiments, the plurality of individual inflatable elements 110a-110d may each be connected to one another, while in other embodiments, the individual inflatable elements 110a-110d may only be connected via the cannula 100. For example, each of the plurality of inflatable members 110a-110d may be individually coupled to the cannula 100 via any suitable connection (e.g., via threaded connection, adhesive, welding, brazing, etc.). In other embodiments, each individual member of the plurality of inflatable members 110a-110d may be coupled to one another (e.g., via bonding, adhesive, etc.) such that a coupling exists between the inflatable members 110a-110d.

In these embodiments, each of the plurality of individual inflatable elements 110a-110d may be positioned circumferentially around the cannula 100 such that each inflatable member is in fluid communication with at least one inflation opening 122. Accordingly, in the present embodiment, the one or more inflation openings may include a plurality of inflation openings, such that each of the plurality of individual inflatable elements are fluidly coupled to the inflation lumen 120 via at least one respective inflation opening. In some embodiments, the plurality of individual inflatable elements 110a-110d may inflate and deflate together. For example, the fluid source 420 may pass fluid into a first member 110a of the plurality of inflatable members 110a-110d, such that fluid passes through the first member 110a and into the remaining members 110b-110d of the plurality of inflatable members, which may cause the plurality of inflatable members 110a-110d to inflate together. In other embodiments, the individual inflatable elements 110a-110d may be individually operated by the pressure source assembly 400. In these embodiments, the pressure source assembly 400 may include a plurality of cannula modules 430, such that each of the individual inflatable elements 110a-110d is controllable by a unique cannula module 430. For example, the pressure source assembly 400 may include four cannula modules 430, such that each cannula module 430 corresponds with one of the individual inflatable elements 110a-110d. In these embodiments, each of the individual inflatable member 110a-110d may be fluidly coupled to a different fluid pump 430a, such that each of the individual inflatable elements 110a-110d may be inflated individually. As the individual inflatable elements 110a-110d inflate, each of the individual inflatable elements 110a-110d may exert an outward pressure P on the biopsy tract. In embodiments in which each of the plurality of individual inflatable elements 110a-110d are inflated individually, each of the individual inflatable elements may exert a unique outward pressure P on different locations within the biopsy tract.

Referring now to FIG. 5, the plurality of individual inflatable elements 110a-110d may each have a predetermined shape in the inflated state. In some embodiments, the individual inflatable elements 110a-110d may each have the same predetermined shape, while in other embodiments, the individual inflatable elements 110a-110d may have different predetermined shapes. The use of individual inflatable elements 110a-110d with different predetermined shapes may be particularly useful when the cannula 100 is being deployed in a biopsy tract that does not have a uniform area, as the use of individual inflatable elements 110a-110d may allow the cannula 100 to better conform to the biopsy tract. In other embodiments still, the individual inflatable elements 110a-110d may be made to conform to the biopsy tract. In these embodiments, the pressure source may provide fluid to each of the series of inflatable members 110a-110d, either individually or simultaneously, until the outward pressure P exerted on the biopsy tract 300 by the plurality of individual inflatable elements 110a-110d reaches a desired level.

Although the cannula 100 embodiment illustrated in FIG. 5 depicts the cannula as having four individual inflatable elements 110a-110d, it should be understood that any number of inflatable members 110 may be deployed on the cannula 100, such that the inflatable members 110 extend from the proximal end 102 to the distal end 104 of the cannula. For example, the plurality of individual inflatable elements 110 may include at least two inflatable members, at least three inflatable members, four or more inflatable members, etc.

Furthermore, the plurality of individual inflatable elements 110 may each have the same cross-sectional shape, while in other embodiments, each of the plurality of individual inflatable elements 110 may have a unique cross-sectional shape. Similarly, the plurality of individual inflatable elements 110 may have various arrangements along the cannula 100. Although the individual inflatable elements 110 are illustrated as covering the entire length LT of the cannula in FIG. 5, the plurality of individual inflatable elements 110 may also be arranged such that gaps exist between the individual inflatable elements 110 along the length of the cannula 100.

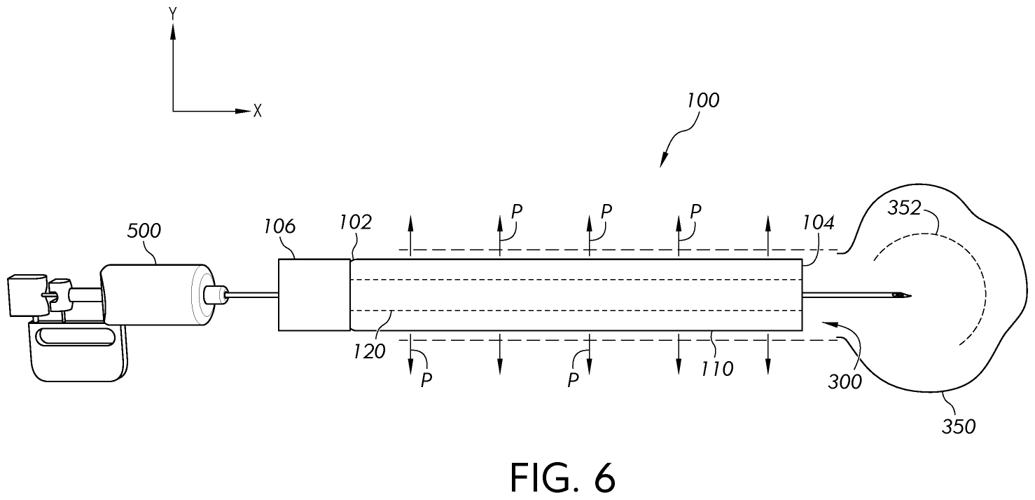

Referring now to FIG. 6, once the cannula 100 is positioned in its desired location within the biopsy tract 300, a user may insert a biopsy needle 500 through the cannula 100 (e.g., in the x+ direction) to acquire a number of biopsy samples from the target site 350. Once the desired number of biopsy samples have been removed from the target site 350, the biopsy needle 500 may be removed from the target site 350 and cannula 100 (e.g., in the x− direction). After the biopsy needle 500 is removed from the cannula 100, the stylet 200 may be inserted through the cannula 100 (e.g., in the x+ direction), such that the second inflatable member 210 extends beyond the distal end 104 of the cannula 100 and into a void 352 in the target site 350 created by the acquisition of the biopsy samples.

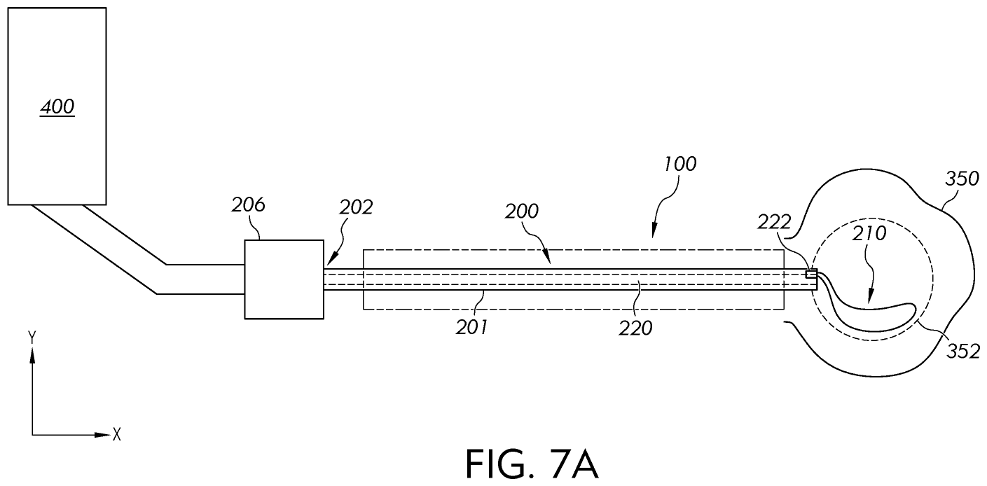

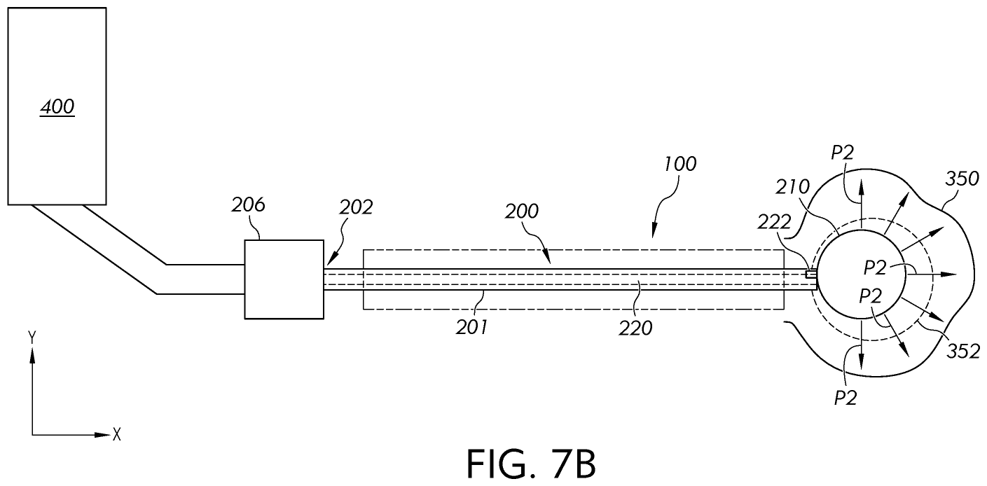

Turning now to FIGS. 7A-7B, the stylet 200 and second inflatable member 210 of the coaxial needle 10 are illustrated in isolation from the cannula 100 and first inflatable member 110. In the depicted embodiment, the second inflatable member 210 is depicted in a second deflated state (FIG. 7A) and a second inflated state (FIG. 7B), respectively. As shown in FIG. 7A, the second deflated state may be defined as a pre-deployed state (e.g., the state prior to the second inflatable member 210 being inflated). In the second deflated state, the second inflatable member 210 and the stylet 200 may be inserted through the cannula 100 and positioned in the target site.

With the cannula 100 positioned in its desired location within the biopsy tract, a user may acquire a number of biopsy samples from the target site. Once the desired number of biopsy samples have been removed from the target site, the stylet 200 may be inserted through the cannula 100, such that the second inflatable member 210 extends beyond the distal end 104 of the cannula 100 and into a void in the target site created by the acquisition of the biopsy samples. With the stylet 200 positioned within the cannula 100, the second inflatable member 210 may be inflated to the second inflated state, as illustrated in FIG. 7B. In these embodiments, the first end 202 of the stylet 200 may be connected to a second inflation port 206, which may provide fluid to the stylet 200. In some embodiments, the first end 202 of the stylet 200 may be molded to the second inflation port 206, while in other embodiments, the first end 202 of the stylet 200 may include a threaded portion which may engage the second inflation port 206.

Referring still to FIG. 7B, the second inflation port 206 may be used to fill the stylet inflation lumen 220 with fluid. As the fluid fills the stylet inflation lumen 220, the fluid may traverse from the first end 202 of the stylet to the second end 204 of the stylet, until the fluid passes through the inflation hole 222 positioned on the second end 204 of the stylet 200. As fluid escapes the inflation hole 222, the second inflatable member 210 may inflate and expand into the void in the target site created by the acquisition of biopsy samples. As the second inflatable member 210 expands to the second inflated state illustrated in FIG. 7B, the second inflatable member 210 may begin to exert an outward pressure P₂ on the void in the target site. The outward pressure P₂ exerted by the second inflatable member 210 on the void may act to stop and/or prevent bleeding within the void created in the target site. More specifically, the outward pressure P₂ exerted on the void may act to slow and/or stop blood flow within the void, which may aid in initiating natural clotting processes within the biopsy void.

In some embodiments, the second inflatable member 210 may be coated with a hemostatic agent to further assist in preventing bleeding within the biopsy tract. In these embodiments, the first inflatable member may be coated with any coating having suitable hemostatic properties, including fibrin, starch, collagen, gelatin, PVA, or the like. It should be understood that the hemostatic coating utilized on the second inflatable member 210 may vary depending on the desired biological result. For example, some hemostatic agents may directly aid in clotting processes, while other hemostatic agents may lead to faster coagulation by absorbing water and blood plasma within the biopsy tract.

Referring still to FIGS. 7A-7B, the pressure source assembly 400 of the coaxial needle 10 may be coupled in fluid communication to the second inflation port 206, such that the stylet module 432 is able to control the volume of fluid which enters the stylet inflation lumen 220, and in turn, the second inflatable member 210. For example, once the stylet 200 is positioned within void 352 created in the target site 350 by the acquisition of tissue samples, the second fluid pump 432a may be operated to cause fluid to enter the stylet inflation lumen 220 such that the second inflatable member 210 is inflated to the second inflated state. As the fluid is pumped from the second fluid pump 432a into the stylet inflation lumen 220 of the stylet 200, the pressure sensor 424 may monitor the outward pressure P₂ applied by the second inflatable member 210 on the void 352 in the target site 350. Once the appropriate amount of pressure P₂ has been achieved, the controller circuit 418 may deactivate the fluid pump 430a in order to prevent additional fluid from entering the inflation lumen 220. When the fluid pump 432a is deactivated, the second valve 432b may be moved from an open position to a closed position, which may further ensure that additional fluid is not passed from the second fluid pump 432a to the stylet inflation lumen 220.

In some embodiments, the second inflatable member 210 may be made to form a predetermined shape in the second inflated state. For example, the second inflatable member 210 may be made to form a sphere, or any other suitable shape, having a predetermined diameter. In these embodiments, the predetermined shape of the second inflatable member 210 may correspond to the size and shape of the biopsy void 352, such that the second inflatable member 210 may apply suitable outward pressure P₂ to the biopsy void 352 in the second inflated state. Although the second inflatable member 210 may be made to form a predetermined shape, it should be understood that the shape of the second inflatable member 210 may not be uniform. For example, the predetermined shape of the second inflatable member 210 may include segments of varying diameters, which may be necessary to apply suitable outward pressure P₂ to the biopsy void 352.

In other embodiments, the second inflatable member 210 may be made to conform to the size and shape of the biopsy void in the second inflated state. In these embodiments, the stylet module 432 of the pressure source assembly 400 may be used to control the inflation of the second inflatable member 210, such that the second inflatable member 210 may exert an optimized amount of outward pressure P₂ on the biopsy void. By optimizing the outward pressure P₂ exerted on the biopsy void, the second inflatable member 210 may maximize the hemostatic benefits provided to the biopsy void without causing additional expansion or irritation of the biopsy void.

In the event excess fluid is supplied to the second inflatable member 210, the outward pressure P₂ applied on the biopsy void may exceed a desired pressure. A desired pressure may be considered the pressure at which the hemostatic benefits of the second inflatable member 210 are optimized, such that second inflatable member 210 aids in hemostasis at the biopsy void without causing damage to the biopsy void. In these embodiments, excessive pressure may result in damage to the biopsy void at the target site 350 and/or the biopsy tract 300. In order to ensure that the outward pressure P₂ does not exceed a desired pressure value, the pressure sensor 424 of the pressure source assembly 400 may monitor the outward pressure P₂ applied on the biopsy void by the second inflatable member 210. When the outward pressure P₂ exceeds the desired pressure value, the vacuum source 422 may activate vacuum pump 422b to apply a vacuum to the stylet inflation lumen 220. As the vacuum is applied to the stylet inflation lumen 220, fluid may be drawn from the second inflatable member 210, thereby alleviating pressure on the biopsy void. Similarly, once the second inflatable member 210 has aided hemostasis within the biopsy void, the vacuum pump 422b may apply a vacuum to the stylet inflation lumen 220 in order to draw fluid out of the second inflatable member 210. As the fluid leaves the second inflatable member 210, the second inflatable member may move from the second inflated state to the second deflated state, such that the stylet 200 may be removed from the cannula 100.

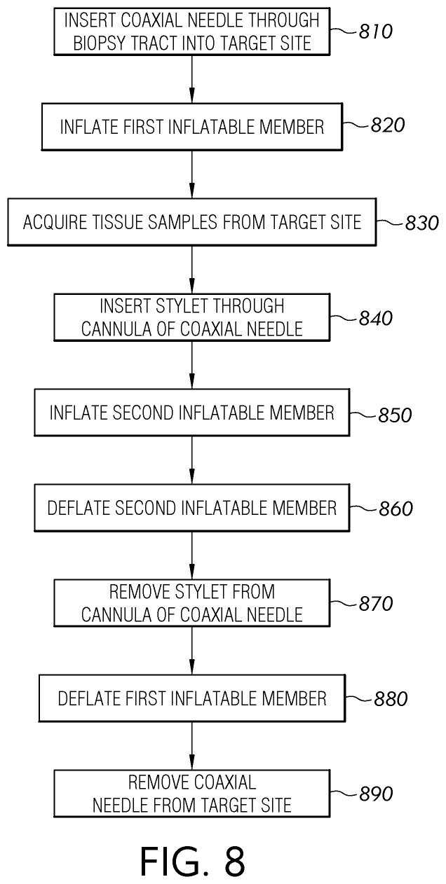

Turning now to FIG. 8, an illustrative method for performing a biopsy procedure using the coaxial needle 10 (FIG. 1) is depicted. As depicted in block 810, the method may first involve inserting the coaxial needle 10 including the cannula 100 into a target site 350 such that a biopsy tract 300 is formed. In these embodiments, the sharpened distal end 104 of the cannula may act to create the biopsy tract 300 as the cannula is inserted into the target site 350 (FIG. 3A). Moving to block 820 of FIG. 8, the method may further include inflating the first inflatable member 110 which surrounds the cannula 100 in a circumferential direction to the inflated position (FIG. 3B).

Referring still to block 820, the first inflatable member 110 may be inflated by filling the first inflatable member 110 with a fluid such as air, saline, or the like, as described above. As the first inflatable member 110 inflates, the first inflatable member 110 may exert an outward pressure P on the biopsy tract 300 which may anchor the first inflatable member 110 in position in the tract 300, as depicted in FIG. 3B. In these embodiments, the first inflatable member 110 may prevent the cannula from moving in a longitudinal or rotational direction. By anchoring the cannula 100 in the biopsy tract 300, it may be possible to prevent or reduce the incidence of irritation or trauma to the biopsy tract 300 as tissue samples are removed from the target site 350. Furthermore, the outward pressure P exerted on the biopsy tract 300 by the first inflatable member 110 may aid in hemostasis within the biopsy tract 300.

With the first inflatable member 110 inflated and the cannula 100 positioned, the method may then move to block 830, which may involve acquiring tissue samples from the target site 350 using a biopsy device, such as a biopsy needle 500, such that a biopsy void 352 is created within the target site, as shown in FIG. 6. Once the desired number of tissue samples have been obtained, the biopsy needle 500 may be removed from the cannula 100, and the method may move to block 840, which may involve inserting the stylet 200 into the cannula 100 of the coaxial needle 10. The stylet 200 may be inserted into and through the cannula 100 such that the second end 204 of the stylet 200 extends beyond the distal end 104 of the cannula 100. With the stylet 200 inserted through the cannula 100 in the position described above, the second inflatable member 210 may extend beyond the distal end 104 of the cannula 100 and into the biopsy void, as shown in FIGS. 7A and 7B.

Once the stylet 200 has been inserted through the cannula 100, the method may involve inflating the second inflatable member 210, as indicated at block 850 and generally depicted in FIG. 7B. The second inflatable member 210 may be inflated by filling the second inflatable member 210 with a fluid such as air, saline, or the like. As the second inflatable member 210 inflates, the second inflatable member may exert an outward pressure P₂ on the biopsy void, which may aid in preventing bleeding within the biopsy void 352.

Turning now to block 860 and block 870 of FIG. 6, once the biopsy void 352 has been treated by the second inflatable member 210, the method may involve deflating the second inflatable member 210 and removing the stylet 200 from the cannula 100. Once the stylet 200 has been removed, the method may move to block 880 and 890, which may involve deflating the first inflatable member 110 of the cannula 100 and removing the coaxial needle 10 from the biopsy tract and target site.

Embodiments may be further described with references to the following numbered clauses:
1. A coaxial needle comprising:
a cannula having a distal end and a proximal end; and
   a first inflatable member surrounding the cannula in a circumferential direction and having a length extending from the distal end to the proximal end of the cannula;
wherein the first inflatable member applies an outward pressure along the entire length of the first inflatable member in an inflated state.
2. The coaxial needle of item 1, wherein the first inflatable member forms a predetermined shape in the inflated state.
3. The coaxial needle of item 1, wherein the first inflatable member is configured to conform to a biopsy tract in the inflated state.
4. The coaxial needle of any of items 1-3, wherein the first inflatable member is filled with air, saline, or other gases in the inflated state.
5. The coaxial needle of any of items 1-4, wherein the first inflatable member is at least partially coated with a hemostatic agent.
6. The coaxial needle of any of items 1-4, further comprising:
a stylet comprising an elongate member having a first end and a second end; and
a second inflatable member located on the second end of the elongate member.
7. The coaxial needle of item 6, wherein the second inflatable member is at least partially coated with a hemostatic agent.
8. The coaxial needle of any of items 6 or 7, wherein the second inflatable conforms to a void left by removal of a tissue sample at a biopsy site in a second inflated state.
9. The coaxial needle of any of items 6-8, wherein the second inflatable member is filled with air, saline, or other gases in the inflated state.
10. The coaxial needle of any of items 1-5, wherein the first inflatable member comprises a single inflatable member.
11. The coaxial needle of any of items 1-5, wherein the first inflatable member comprises a plurality of individual inflatable elements.
12. The coaxial needle of any of items 1-5, further comprising a first fluid pump and a vacuum pump coupled in fluid communication with the first inflatable member, the first fluid pump and vacuum pump configured to alternate the first inflatable member between the inflated state and a deflated state.
13. The coaxial needle of any of items 6-9 further comprising a second fluid pump and a vacuum pump coupled in fluid communication with the second inflatable member, the second fluid pump and vacuum pump configured to alternate the second inflatable member between a second inflated state and a second deflated state.
14. A coaxial needle comprising:
   a cannula having a distal end and a proximal end;
      a first inflatable member surrounding the cannula in a circumferential direction and having a length extending from the distal end to the proximal end of the cannula, the first inflatable member having a first inflated state and a first deflated state; and
      a stylet comprising an elongate member having a first end and a second end; and wherein the stylet includes
   a second inflatable member coupled to the second end of the elongate member, the second inflatable member having a second inflated state and a second deflated state.
15. The coaxial needle of item 14, wherein the first inflatable member applies an outward pressure along its entire length in the first inflated state.
16. The coaxial needle of any of items 14 or 15, wherein the second inflatable member applies outward pressure to a void left by removal of a tissue sample at a biopsy site in the second inflated state.
17. The coaxial needle of any of items 14-16, wherein the first inflatable member is made to form a predetermined shape in the first inflated state.
18. The coaxial needle of any of items 14-17, wherein at least one of the first inflatable member and the second inflatable member is coated with a hemostatic agent.
19. A method of performing a biopsy comprising:
   inserting a coaxial needle into a target site, the coaxial needle including a cannula having a proximal end and a distal end;
   inflating a first inflatable member which surrounds the coaxial needle in a circumferential direction and extends from the proximal end of the cannula to the distal end of the cannula;
   inserting a biopsy needle in the cannula of the coaxial needle and acquiring samples from the target site, such that a biopsy void is created in the target site;
   removing the biopsy needle from the cannula of the coaxial needle
   inserting a stylet that includes an elongate member having a first end and a second end in the cannula of the coaxial needle; and
   inflating a second inflatable member located on the second end of the elongate member of the stylet in the biopsy void created in the target site.
20. The method of item 19, further comprising:
deflating the second inflatable member of the stylet;
   removing the stylet from the cannula;
   deflating the first inflatable member of the coaxial needle; and
   removing the coaxial needle from the target site.

As should be appreciated in view of the foregoing, a coaxial needle is described herein. The coaxial needle may include a cannula having a distal end and a proximal end. A first inflatable member may surround the cannula in a circumferential direction and have a length which extends from the proximal end of the cannula to the distal end of the cannula. The first inflatable member may apply an outward pressure along its entire length in an inflated state. In these embodiments, the first inflatable member may apply outward pressure on a biopsy tract, such that the cannula may become anchored in the biopsy tract. Securing the coaxial needle in a desired anatomical position may alleviate the risk of the needle backing out or moving along the biopsy tract. Furthermore, the pressure applied by the inflatable member of the coaxial needle on the biopsy tract may aid in hemostasis. plurality of individual

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A coaxial needle comprising:
a cannula having a distal end and a proximal end; and
a first inflatable member surrounding the cannula in a circumferential direction and having a length extending from the distal end to the proximal end of the cannula;
wherein the first inflatable member applies an outward pressure along the entire length of the first inflatable member in an inflated state.

2. The coaxial needle of claim 1, wherein the first inflatable member forms a predetermined shape in the inflated state.

3. The coaxial needle of claim 1, wherein the first inflatable member is configured to conform to a biopsy tract in the inflated state.

4. The coaxial needle of claim 1, wherein the first inflatable member is filled with air, saline, or other gases in the inflated state.

5. The coaxial needle of claim 1, wherein the first inflatable member is at least partially coated with a hemostatic agent.

6. The coaxial needle of claim 1, further comprising:
a stylet comprising an elongate member having a first end and a second end; and
a second inflatable member located on the second end of the elongate member.

7. The coaxial needle of claim 6, wherein the second inflatable member is at least partially coated with a hemostatic agent.

8. The coaxial needle of claim 6, wherein the second inflatable member conforms to a void left by removal of a tissue sample at a biopsy site in a second inflated state.

9. The coaxial needle of claim 6, wherein the second inflatable member is filled with air, saline, or other gases in the inflated state.

10. The coaxial needle of claim 1, wherein the first inflatable member comprises a single inflatable member.

11. The coaxial needle of claim 1, wherein the first inflatable member comprises a plurality of individual inflatable elements.

12. The coaxial needle of claim 1, further comprising a first fluid pump and a vacuum pump coupled in fluid communication with the first inflatable member, the first fluid pump and vacuum pump configured to alternate the first inflatable member between the inflated state and a deflated state.

13. The coaxial needle of claim 6, further comprising a second fluid pump and a vacuum pump coupled in fluid communication with the second inflatable member, the second fluid pump and vacuum pump configured to alternate the second inflatable member between a second inflated state and a second deflated state.

14. A coaxial needle comprising:
a cannula having a distal end and a proximal end;
a first inflatable member surrounding the cannula in a circumferential direction and having a length extending from the distal end to the proximal end of the cannula, the first inflatable member having a first inflated state and a first deflated state; and
a stylet comprising:
an elongate member having a first end and a second end, and
a second inflatable member coupled to the second end of the elongate member, the second inflatable member having a second inflated state and a second deflated state.

15. The coaxial needle of claim 14, wherein the first inflatable member applies an outward pressure along its entire length in the first inflated state.

16. The coaxial needle of claim 15, wherein the second inflatable member applies outward pressure to a void left by removal of a tissue sample at a biopsy site in the second inflated state.

17. The coaxial needle of claim 14, wherein the first inflatable member forms a predetermined shape in the first inflated state.

18. The coaxial needle of claim 14, wherein at least one of the first inflatable member and the second inflatable member is coated with a hemostatic agent.

19. A method of performing a biopsy comprising:
inserting a coaxial needle into a target site, the coaxial needle including a cannula having a proximal end and a distal end;
inflating a first inflatable member which surrounds the coaxial needle in a circumferential direction and extends from the proximal end of the cannula to the distal end of the cannula;
inserting a biopsy needle in the cannula of the coaxial needle and acquiring samples from the target site, such that a biopsy void is created in the target site;
removing the biopsy needle from the cannula of the coaxial needle inserting a stylet that includes an elongate member having a first end and a second end in the cannula of the coaxial needle; and
inflating a second inflatable member located on the second end of the elongate member of the stylet in the biopsy void created in the target site.

20. The method of claim 19, further comprising:
deflating the second inflatable member of the stylet;
removing the stylet from the cannula;
deflating the first inflatable member of the coaxial needle; and
removing the coaxial needle from the target site.
